SEQUENCE LISTING (11 sequences)

SEQ ID NO: 1 - protein, 12 aa, synthetic construct
NYSKPTDRQYHF

SEQ ID NO: 2 - DNA, 22 bp, synthetic construct
  source 1..22: mol_type = other DNA
tcaacatcagtctgataagcta

SEQ ID NO: 3 - DNA, 22 bp, synthetic construct
  source 1..22: mol_type = other DNA
  modified_base 1^2: mod_base = OTHER; note = Phosphorothioate linkage
  modified_base 2: mod_base = OTHER; note = 2,4-bicyclics with O-methylene bridge of cytosine
  modified_base 2^3: mod_base = OTHER; note = Phosphorothioate linkage
  modified_base 3^4: mod_base = OTHER; note = Phosphorothioate linkage
  modified_base 5: mod_base = OTHER; note = 2,4-bicyclics with O-methylene bridge of cytosine
  modified_base 8: mod_base = OTHER; note = 2,4-bicyclics with O-methylene bridge of cytosine
  modified_base 11: mod_base = OTHER; note = 2,4-bicyclics with O-methylene bridge of thymine
  modified_base 14: mod_base = OTHER; note = 2,4-bicyclics with O-methylene bridge of guanine
  modified_base 17: mod_base = OTHER; note = 2,4-bicyclics with O-methylene bridge of adenine
  modified_base 19^20: mod_base = OTHER; note = Phosphorothioate linkage
  modified_base 20: mod_base = OTHER; note = 2,4-bicyclics with O-methylene bridge of cytosine
  modified_base 20^21: mod_base = OTHER; note = Phosphorothioate linkage
  modified_base 21^22: mod_base = OTHER; note = Phosphorothioate linkage
tcaacatcagtctgataagcta

SEQ ID NO: 4 - RNA, 23 nt, synthetic construct
  misc_feature 1..23: note = RNA
  source 1..23: mol_type = other RNA
aaccctatcacgattagcattaa

SEQ ID NO: 5 - RNA, 22 nt, synthetic construct
  source 1..22: mol_type = other RNA
tgtagtgcaactatgcaaaact

SEQ ID NO: 6 - RNA, 22 nt, synthetic construct
  source 1..22: mol_type = other RNA
tgaaatgcaaacctgcaaaact

SEQ ID NO: 7 - RNA, 23 nt, synthetic construct
  source 1..23: mol_type = other RNA
ctacctgcactgtaagcactttg

SEQ ID NO: 8 - RNA, 22 nt, synthetic construct
  source 1..22: mol_type = other RNA
tcacaagttagggtctcaggga

SEQ ID NO: 9 - RNA, 22 nt, synthetic construct
  source 1..22: mol_type = other RNA
gatagagtgcagaccagggtct

SEQ ID NO: 10 - RNA, 21 nt, synthetic construct
  source 1..21: mol_type = other RNA
caattgcccaagtctccgcct

SEQ ID NO: 11 - RNA, 22 nt, synthetic construct
  source 1..22: mol_type = other RNA
cttccagtcggggatgtttaca

(19) United States
(12) Patent Application Publication
(10) Pub. No.: US 2024/0189435 A1
(43) Pub. Date: Jun. 13, 2024

(54) NOVEL PEPTIDE-OLIGONUCLEOTIDE CONJUGATE AND USE THEREOF

(71) Applicant: KOREA INSTITUTE OF SCIENCE AND TECHNOLOGY, Seoul (KR)

(72) Inventors: Yoo Soo YANG, Seoul (KR); Sun Hwa KIM, Seoul (KR); Ick Chan KWON, Seoul (KR); Eun Hye KIM, Seoul (KR)

(73) Assignee: KOREA INSTITUTE OF SCIENCE AND TECHNOLOGY, Seoul (KR)

(21) Appl. No.: 18/551,271

(22) PCT Filed: Dec. 13, 2022
(86) PCT No.: PCT/KR2022/020176

§ 371 Date: Sep. 19, 2023

(30) Foreign Application Priority Data
Dec. 22, 2021 (KR) 10-2021-0184545
Dec. 12, 2022 (KR) 10-2022-0173072

(51) Int. Cl. A61K 47/64; A61K 47/54; A61P 35/00; C12N 15/11

(52) U.S. Cl. CPC A61K 47/64; A61K 47/545; A61P 35/00; C12N 15/11; C12N 2310/11; C12N 2310/14; C12N 2310/141; C12N 2310/16; C12N 2310/315; C12N 2310/3231; C12N 2310/351; C12N 2310/3529; C12N 2310/531; C12N 2330/00

(57) ABSTRACT

Disclosed is an agent capable of inhibiting the activity or enhancing expression of various cancer-related RNAs in TAMS and cancer cells. Disclosed is also a dual-targeted drug delivery system capable of binding to both tumor cells and macrophages in which the PD-L1 receptor is overexpressed.

### TECHNICAL FIELD

The present invention relates to a novel peptide-oligonucleotide conjugate capable of targeting both cancer cells and macrophages overexpressing a PD-L1 receptor and the use thereof.

### BACKGROUND ART

### Disclosure

MicroRNAs (miRNAs) are small endogenous non-coding RNAs including 20 to 25 nucleotides that control the expression of several genes in various cells and regulate many physiological cellular processes and pathologies. In addition, specific miRNAs within the tumor microenvironment (TME) may be used as potential cancer biomarkers or therapeutic targets, since widespread dysregulation of miRNA expression within the TME is strongly associated with tumor regression.

miRNA-21 (miR-21), which is one of the first identified oncogenic miRNAs, is commonly up-regulated in various types of tumors, including melanoma, and generally has a poor survival prognosis. For example, miR-21 knockout mice have been reported to develop smaller B16 tumors than wild-type mice. miR-21 is involved in tumorigenesis by regulating various downstream effectors involved in tumorigenesis, including proliferation, apoptosis, cell invasion, migration and chemoresistance. miR-21 is the most abundantly expressed miRNA in TME-infiltrating non-malignant cells as well as cancer cells, and regulates cell functions during tumorigenesis. In particular, the expression of miR-21 in tumor-associated macrophages (TAMs) plays a pivotal role in regulating macrophage polarization for the pro-tumorigenic M2-like phenotypes. M2-polarized TAMs enhance tumor metastasis, promote angiogenesis and interfere with adaptive immune responses to promote tumor growth. In particular, expression of miR-21 in TAMs has been reported to be responsible for promoting tumor growth.

miR-155, which is a representative tumor-promoting miRNA, has been reported to have oncogenic miRNA characteristics that are overexpressed in various hematological cancers and solid cancers including breast cancer, head and neck cancer, lymphoma and a variety of solid tumors.

miR-19a/b, which was the first miRNA identified to be oncogenic, is the most key miRNA among the oncogenic miRNA 17-92 clusters. In particular, it has been reported to be overexpressed in various types of cancers, including acute myeloid leukemia, colorectal cancer and gastric cancer, and functions to promote tumor growth and metastasis.

miR-17-92 is potent tumor-inducing miRNA that has been researched most thoroughly and has been reported to play a key role in various processes such as the cell cycle, proliferation, and apoptosis.

Abnormal expression of miR-128 has been reported to be found in many types of malignant tumors.

miR-125b is overexpressed in various carcinomas including non-small-cell lung cancer (NSCLC), acute myeloid leukemia (AML), and gastric cancer. In particular, research has reported that miR-125b functions to have not only oncogenicity but also antitumor activity under tumor microenvironments in different carcinomas.

miR-504 has been reported as oncogenic miRNA that promotes cell-cycle arrest, apoptosis, and tumorigenesis involving FOXP1, NRF1, CDK6, and P53, which are mRNAs studied as tumor suppressor genes, by directly interacting with and regulating the genes.

miR-25 is reported to be overexpressed in various carcinomas including gastric cancer, prostate cancer, liver cancer, and colon cancer.

miR-30d is a member of the miR-30 family responsible for tumor development and progression, and the abnormal expression thereof has been reported to be associated with tumor suppressor genes or oncogenes in the onset and progression of various cancers.

Targeting both tumor cells and TAMs rather than killing the tumor cells alone has proven to be a promising strategy to improve the efficacy of anti-tumor therapies. However, studies on antitumor therapeutics targeting both tumor cells and TAMs are still insufficient.

Meanwhile, anti-miRNA oligonucleotides (anti-miRNAs) are potential therapeutic agents for cancer for effective and irreversible suppression of abnormal miRNA expression. However, anti-miRNAs are readily removed by the reticuloendothelial system and are readily degraded in serum. The most important issue is that charge repulsion disrupts the interaction between the anti-miRNA and cell membrane, resulting in poor cellular uptake. Therefore, anti-miRNA-based therapy should be supported by an appropriate delivery system.

### PRIOR ART LITERATURE

### Patent Document

(Patent Document 1) Korea Patent No. 10-0721696

### DISCLOSURE

### Technical Problem

Accordingly, as a result of research to solve the problems described above, the present inventors designed a peptide-oligonucleotide conjugate having a structure in which anti-miRNA is directly bound to a PD-L1-binding peptide. It is an object of the present invention to provide such a peptide-oligonucleotide conjugate.

The objects of the present invention are not limited to those described above. The objects of the present invention will be clearly understood from the following description and are capable of being implemented by means defined in the claims and combinations thereof.

### Technical Solution

In order to accomplish the objects of the present invention, the present invention provides solutions to the following object.

In one aspect, the present invention provides a peptide-oligonucleotide conjugate, wherein the peptide is a PD-L1-binding peptide, and the oligonucleotide is an oligonucleotide or a modified oligonucleotide thereof.

In one aspect of the present invention, the PD-L1-binding peptide may have a sequence of SEQ ID NO: 1.

In one aspect of the present invention, the PD-L1-binding peptide may be a PD-L1-binding peptide functionalized with an azide group.

In one aspect of the present invention, the oligonucleotide may be small interfering RNA (siRNA), small hairpin RNA (shRNA), miRNA, an antisense oligonucleotide or a nucleic acid aptamer.

In one aspect of the present invention, the oligonucleotide may be a miRNA inhibitor including at least one of anti-miRNA 21, anti-miRNA 155, anti-miRNA 19, anti-miRNA 17-92, anti-miRNA 128, anti-miRNA 125b, anti-miRNA 504, anti-miRNA 25, and anti-miRNA 30d.

In an embodiment, the miRNA inhibitor may include at least one of anti-miRNA 21, anti-miRNA 155, anti-miRNA 19, anti-miRNA 17-92, and anti-miRNA 128.

In one aspect of the present invention, the oligonucleotide may be a miRNA carrier including at least one of miRNA 34a, miRNA 194, miRNA 192, miRNA 29, miRNA 215, miRNA 200, miRNA 605, miRNA 122 and miRNA 143/145.

In an embodiment, the miRNA carrier may include at least one of miRNA 34a, miRNA 194, miRNA 192, and miRNA 29.

In one aspect of the present invention, the modified oligonucleotide thereof may include at least one of a modified internucleoside linkage, a modified sugar, and a modified nucleobase.

In an embodiment, the modified internucleoside linkage may include at least one of a phosphorothioate (PS) internucleoside linkage, a phosphodiester internucleoside linkage, a phosphotriester internucleoside linkage, a morpholino internucleoside linkage, and a protein nucleic acid (PNA) internucleoside linkage, and the modified sugar may include at least one of 2′-O-methoxyethyl, 2′-O-hydroxymethyl, 2′-hydroxyl, 2′-fluoro, and 2′,4′-LNA.

In an embodiment, the modified internucleoside linkage may be a phosphorothioate (PS) internucleoside linkage, and the modified sugar may be 2′,4′-LNA.

In one aspect of the present invention, the oligonucleotide may be anti-miRNA-21.

In one aspect of the present invention, the anti-miRNA-21 may have a sequence of SEQ ID NO: 2.

In one aspect of the present invention, the oligonucleotide may be modified anti-miRNA-21, the modified anti-miRNA-21 may include 1 to 10 phosphorothioate (PS) internucleoside linkages in an entire sequence of the oligonucleotide, and the modified anti-miRNA-21 may include 1 to 10 modified sugars of 2′,4′-LNA in the entire sequence of the oligonucleotide.

In an embodiment, the modified anti-miRNA-21 may be represented by the following sequence structural formula, wherein PS represents a phosphorothioate (PS) internucleoside linkage, and the subscript L represents a modified sugar of 2′,4′-LNA.

5′-
T(PS)C_{L}(PS)A(PS)AC_{L}ATC_{L}AGT_{L}CTG_{L}ATA_{L}AG(PS)C_{L}(PS)T(PS)
A-3′

In an embodiment, the sequence structural formula may be represented by a sequence of SEQ ID NO: 3.

In one aspect of the present invention, the oligonucleotide may be an oligonucleotide functionalized with diarylcyclooctyne (DBCO).

In one aspect of the present invention, the peptide-oligonucleotide conjugate may be a conjugate in which the PD-L1-binding peptide functionalized with the azide group is bound to the oligonucleotide functionalized with diarylcyclooctyne (DBCO) by a click reaction.

In one aspect of the present invention, the peptide-oligonucleotide conjugate may have a chemical structure represented by the following Formula 1:

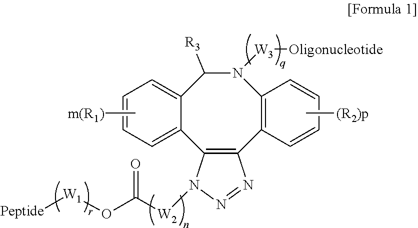

wherein
R₁ to R₃ are each independently hydrogen, a C₁-C₁₃ alkyl group, a C₁-C₆ alkoxy group, a C₆-C₁₀ aryl group, a C₃-C₁₀ cyclyl group, a C₃-C₁₀ heteroaryl group, a C₃-C₁₀ heterocyclyl group, —C(O)—(C₁-C₁₃ alkyl), —C(O)—(C₆-C₁₀ aryl), or —C(O)—(C₃-C₁₀ heteroaryl);
m, n, p, q or r each independently represents 0, 1, 2, 3 or 4;
W₁ to W₃ each independently represent —NR₃—, —NR₃CH₂—, —NR₃—C(O)—, —NR₃—C(O)—NR₄—, —NR₃—C(S)—NR₄—, —C(O)—, —C(O)CH₂—, —C(O)O—, —C(O)NR₃—, —(CH₂)—, —(CR₃R₄)—, —(CH₂)(CR₃R₄)—, —S(O)₂—, —NR₃S(O)₂—, or —S(O)₂NR₃—;
the C₁-C₁₃ alkyl group, the C₃-C₁₀ cyclyl group, and the C₁-C₆ alkoxy group include at least one substituent selected from the group consisting of hydrogen, a hydroxyl group, a halogen group, a C₁-C₁₃ alkyl group, a C₁-C₆ alkoxy group, an amino group (—NR₃R₄), a nitro group (—N(O)₂), an amide group (—(C═O)NR₃R₄), a carboxylic acid group (—C(O)OH), a nitrile group (—CN), a urea group (—NR₃(C═O)NR₄—), a sulfonamide group (—NHS(O)₂—), a sulfide group (—S—), a sulfone group (—S(O)₂—), a phosphoryl group (—P(O)R₃R₄), a C₆-C₁₀ aryl group, a C₃-C₁₀ heteroaryl group, and a C₃-C₁₀ heterocyclyl group,
the C₆-C₁₀ aryl group, the C₃-C₁₀ heteroaryl group or the C₃-C₁₀ heterocyclyl group includes at least one substituent selected from the group consisting of hydrogen, a hydroxyl group, a halogen group, a carbonyl group (—(C═O)R₃R₄), a C₁-C₃ alkyl group unsubstituted or substituted with a halogen or C₃-C₁₀ heterocyclyl group, a C₁-C₃ alkoxy group unsubstituted or substituted with a halogen or C₃-C₁₀ heterocyclyl group, C₆-C₁₀ phenoxy, an amino group (—NR₃R₄), a nitro group (—N(O)₂), an amide group (—(C═O)NR₃R₄), a carboxylic acid group (—C(O)OH), a nitrile group (—CN), a urea group (—NR₃(C═O)NR₄—), a sulfonamide group (—NHS(O)₂—), a sulfide group (—S—), a sulfone group (—S(O)₂—), a phosphoryl group (—P(O)R₃R₄), a C₆-C₁₀ aryl group, a C₃-C₁₀ heteroaryl group, and a C₃-C₁₀ heterocyclyl group; and
R₃ and R₄ are each independently hydrogen, a C₁-C₆ alkyl group, a C₁-C₆ alkenyl group, a C₁-C₆ alkynyl group, a C₆-C₁₀ aryl group, a C₃-C₁₀ heteroaryl group, or a C₃-C₁₀ heterocyclyl group, or R₃ optionally includes at least one of N, O, S, NH, C═N, C═O, —NHC(O)—, —NHC(O)NH—, —NHS(O)₂—, or SO₂ along with a nitrogen or carbon atom bound to R₄, and forms a 3- to 7-membered saturated ring that is optionally substituted with at least one of hydrogen, a C₁-C₁₃ alkyl group, a C₆-C₁₀ aryl group, a C₃-C₁₀ heteroaryl group, a hydroxyl group, a halide group, and a cyano group, and the C₃-C₁₀ heteroaryl group and the C₃-C₁₀ heterocyclyl group include at least one heteroatom selected from the group consisting of N, O, and S.

In an embodiment,
R₁ or R₂ may each independently represent hydrogen, or a C₁-C₁₃ alkyl group; m or p may each independently represent 0, 1, 2, 3 or 4;
R₃ may be hydrogen or a C₁-C₃ alkyl group;
W₁ may be —(CH₂)—, —(CR₃R₄)—, or —(CH₂)(CR₃R₄)—; r may be 0, 1 or 2;
W₂ may be —(CH₂)—, or —(CR₃R₄)—; n may be 1 or 2;
W₃ may be —(CH₂)—, or —(CR₃R₄)—; and q may be 0, 1 or 2.

In one aspect of the present invention, in Formula 1, the peptide may be a PD-L1-binding peptide, and the PD-L1-binding peptide may have a sequence of SEQ ID NO: 1, and in Formula 1, the oligonucleotide may be anti-miRNA-21 or modified anti-miRNA-21, the anti-miRNA-21 may have a sequence of SEQ ID NO: 2, and the modified anti-miRNA-21 may have a sequence of SEQ ID NO: 3.

In one aspect of the present invention, the oligonucleotide may be any one of the following: anti-miRNA 155 represented by SEQ ID NO: 4; anti-miRNA 19a represented by SEQ ID NO: 5; anti-miRNA 19b represented by SEQ ID NO: 6; anti-miRNA 17 represented by SEQ ID NO: 7; anti-miRNA 125b represented by SEQ ID NO: 8; anti-miRNA 504 represented by SEQ ID NO: 9; anti-miRNA 25 represented by SEQ ID NO: 10; and anti-miRNA 30d represented by SEQ ID NO: 11.

In one aspect of the present invention, the peptide-oligonucleotide conjugate may inhibit expression of at least one of miR-21, miRNA 155, miRNA 19, miRNA 17-92, miRNA 128, miRNA 125b, miRNA 504, miRNA 25, and miRNA 30d in cells.

In one aspect of the present invention, the peptide-oligonucleotide conjugate may improve expression of at least one of miRNA 34a, miRNA 194, miRNA 192, miRNA 29, miRNA 215, miRNA 200, miRNA 605, miRNA 122, and miRNA 143/145 in cells.

In another aspect, the present invention provides a pharmaceutical formulation containing the peptide-oligonucleotide conjugate according to one aspect of the present invention.

In another aspect, the present invention provides a pharmaceutical formulation for preventing, ameliorating or treating cancer containing the peptide-oligonucleotide conjugate according to one aspect of the present invention.

The cancer may be melanoma, breast cancer, lung cancer, colon cancer, ovarian cancer, head and neck cancer, or neuroblastoma.

In another aspect, the present invention provides a method for preparing the peptide-oligonucleotide conjugate according to one aspect, the method including (a) functionalizing PD-L1-binding peptide with an azide group, (b) functionalizing oligonucleotide with diarylcyclooctyne (DBCO), and (c) reacting oligonucleotide functionalized with diarylcyclooctyne (DBCO) with the PD-L1-binding peptide functionalized with the azide group by a click reaction to prepare a peptide-oligonucleotide conjugate.

The PD-L1-binding peptide functionalized with the azide group may be a compound represented by the following Formula 2, and the oligonucleotide functionalized with diarylcyclooctyne (DBCO) may be a compound represented by the following Formula 3:

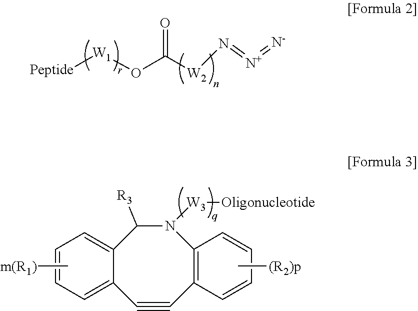

wherein
R₁ to R₃ each independently represent hydrogen, a C₁-C₁₃ alkyl group, a C₁-C₆ alkoxy group, a C₆-C₁₀ aryl group, a C₃-C₁₀ cyclyl group, a C₃-C₁₀ heteroaryl group, a C₃-C₁₀ heterocyclyl group, —C(O)—(C₁-C₁₃ alkyl), —C(O)—(C₆-C₁₀ aryl), or —C(O)—(C₃-C₁₀ heteroaryl);
m, n, p, q or r each independently represents 0, 1, 2, 3 or 4;
W₁ to W₃ each independently represent —NR₃—, —NR₃CH₂—, —NR₃—C(O)—, —NR₃—C(O)—NR₄—, —NR₃—C(S)—NR₄—, —C(O)—, —C(O)CH₂—, —C(O)O—, —C(O)NR₃—, —(CH₂)—, —(CR₃R₄)—, —(CH₂)(CR₃R₄)—, —S(O)₂—, —NR₃S(O)₂—, or —S(O)₂NR₃;
wherein the C₁-C₁₃ alkyl group, the C₃-C₁₀ cyclyl group, and the C₁-C₆ alkoxy group include at least one substituent selected from the group consisting of hydrogen, a hydroxyl group, a halogen group, a C₁-C₁₃ alkyl group, a C₁-C₆ alkoxy group, an amino group (—NR₃R₄), a nitro group (—N(O)₂), an amide group (—(C═O)NR₃R₄), a carboxylic acid group (—C(O)OH), a nitrile group (—CN), a urea group (—NR₃(C═O)NR₄—), a sulfonamide group (—NHS(O)₂—), a sulfide group (—S—), a sulfone group (—S(O)₂—), a phosphoryl group (—P(O)R₃R₄), a C₆-C₁₀ aryl group, a C₃-C₁₀ heteroaryl group, and a C₃-C₁₀ heterocyclyl group,
the C₆-C₁₀ aryl group, the C₃-C₁₀ heteroaryl group or the C₃-C₁₀ heterocyclyl group includes at least one substituent selected from the group consisting of hydrogen, a hydroxyl group, a halogen group, a carbonyl group (—(C═O)R₃R₄), a C₁-C₃ alkyl group unsubstituted or substituted with a halogen or C₃-C₁₀ heterocyclyl group, a C₁-C₃ alkoxy group unsubstituted or substituted with a halogen or C₃-C₁₀ heterocyclyl group, C₆-C₁₀ phenoxy, an amino group (—NR₃R₄), a nitro group (—N(O)₂), an amide group (—(C═O)NR₃R₄), a carboxylic acid group (—C(O)OH), a nitrile group (—CN), a urea group (—NR₃(C═O)NR₄—), a sulfonamide group (—NHS(O)₂—), a sulfide group (—S—), a sulfone group (—S(O)₂—), a phosphoryl group (—P(O)R₃R₄), a C₆-C₁₀ aryl group, a C₃-C₁₀ heteroaryl group, and a C₃-C₁₀ heterocyclyl group; and
R₃ and R₄ each independently represent hydrogen, a C₁-C₆ alkyl group, a C₁-C₆ alkenyl group, a C₁-C₆ alkynyl group, a C₆-C₁₀ aryl group, a C₃-C₁₀ heteroaryl group, or a C₃-C₁₀ heterocyclyl group, or R₃ optionally includes at least one of N, O, S, NH, C═N, C═O, —NHC(O)—, —NHC(O)NH—, —NHS(O)₂—, or SO₂ along with a nitrogen or carbon atom bound to R₄, and forms a 3- to 7-membered saturated ring that is optionally substituted with at least one of hydrogen, a C₁-C₁₃ alkyl group, a C₆-C₁₀ aryl group, a C₃-C₁₀ heteroaryl group, a hydroxyl group, a halide group, and a cyano group, and the C₃-C₁₀ heteroaryl group and the C₃-C₁₀ heterocyclyl group include at least one heteroatom selected from the group consisting of N, O, and S.

In an embodiment, R₁ or R₂ may each independently represent hydrogen, or a C₁-C₁₃ alkyl group; m or p each independently represents 0, 1, 2, 3 or 4; R₃ is hydrogen, or a C₁-C₃ alkyl group; W₁ is —(CH₂)—, —(CR₃R₄)—, or —(CH₂)(CR₃R₄)—; r is 0, 1 or 2; W₂ is —(CH₂)—, or —(CR₃R₄)—; n is 1 or 2; W₃ is —(CH₂)—, or —(CR₃R₄)—; and q is 0, 1 or 2.

In another aspect, the click reaction in step (c) may be performed in a pH 6.5-8.0 buffer solution at 700 rpm to 1,500 rpm and a temperature of 30° C. to 45° C.

In another aspect, in step (c), the oligonucleotide functionalized with diarylcyclooctyne (DBCO) and the PD-L1-binding peptide functionalized with the azide group may be synthesized at a molar ratio of 1:1.5 to 1:2.5.

In another aspect, in step (C), the reaction may be performed for 1 to 3 hours.

In another aspect, the PD-L1-binding peptide may have a sequence of SEQ ID NO: 1.

In another aspect, the anti-miRNA-21 may have a sequence of SEQ ID NO: 2 or SEQ ID NO: 3.

### Advantageous Effects

The peptide-oligonucleotide conjugate according to one aspect of the present invention targets both cancer cells and macrophages that overexpress the PD-L1 receptor.

The peptide-oligonucleotide conjugate according to one aspect of the present invention binds to both cancer cells and macrophages in which miRNA-21 (miR-21), miRNA 155, miRNA 19, miRNA 17-92, miRNA 128, miRNA 125b, miRNA 504, miRNA 25 and miRNA 30d are expressed to regulate the expression of the nucleic acids.

The peptide-oligonucleotide conjugate according to one aspect of the present invention enables effective treatment of various cancers including melanoma, breast cancer, lung cancer, colon cancer, ovarian cancer, head and neck cancer, and neuroblastoma.

The effects of the present invention are not limited to those mentioned above. It should be understood that the effects of the present invention include all effects that can be inferred from the following description.

### DESCRIPTION OF DRAWINGS

FIG. 1 is a schematic diagram illustrating a synthesis of Example 1 of the present invention.

FIG. 2 shows the results of a test to compare the molar ratio in Example 2.

FIG. 3 shows the results of the test to compare the conjugation time in Example 2.

FIG. 4 shows the result of Confirmation-1 of binding affinity between miRNA-21 and Pep-21 in Example 3.

FIG. 5 shows the result of Confirmation-2 of binding affinity between miRNA-21 and Pep-21 in Example 3.

FIG. 6 shows the result of the test in Example 4.

FIG. 7 shows the result in Example 5.

FIG. 8 shows the result of test in Example 6-1.

FIGS. 9A and 9B show the results of test in Example 6-2.

FIG. 10 shows the result of the test in Example 7.

FIG. 11A is a schematic diagram illustrating an animal test in Example 8, and FIGS. 11B and 11C show the results of the test in Example 8.

FIGS. 12A and 12B show the results of TUNEL assay in Example 9.

FIGS. 12C and 12D show the results of immunofluorescence assay in Example 9.

FIG. 13 shows the result of the test in Example 10.

FIG. 14A shows the result of RT-PCR in Example 11, FIG. 14B shows the result of toxicity test in Example 11, and FIG. 14C shows the result of western blotting in Example 11.

FIG. 15 shows the result of test in Example 12.

FIGS. 16A and 16B show the results of test in Example 13.

FIG. 17 is a schematic diagram illustrating the configuration of a drug delivery system according to an embodiment of the present invention.

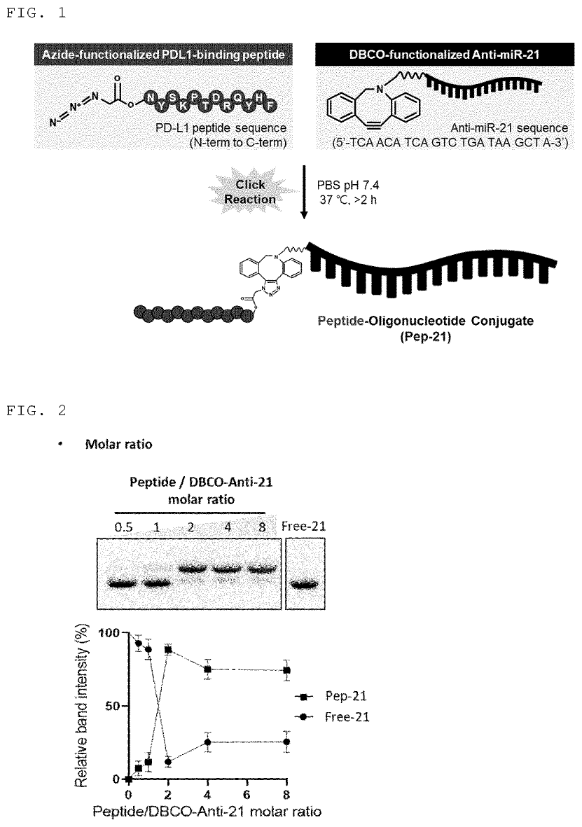

### BEST MODE

Unless the context clearly indicates otherwise, all numbers, figures and/or expressions that represent ingredients, reaction conditions, polymer compositions, and amounts of mixtures used in the specification are approximations that reflect various uncertainties of measurement occurring inherently in obtaining these figures, among other things. For this reason, it should be understood that, in all cases, the term “about” should be understood to modify all numbers, figures, and/or expressions. In addition, when numerical ranges are disclosed in the description, these ranges are continuous and include all numbers from the minimum to the maximum, including the maximum within each range, unless otherwise defined. Furthermore, when the range refers to an integer, it includes all integers from the minimum to the maximum, including the maximum within the range, unless otherwise defined.

It should be understood that, in the specification, when a range is referred to regarding a parameter, the parameter encompasses all figures including end points disclosed within the range. For example, the range of “5 to 10” includes figures of 5, 6, 7, 8, 9, and 10, as well as arbitrary sub-ranges, such as ranges of 6 to 10, 7 to 10, 6 to 9, and 7 to 9, and any figures, such as 5.5, 6.5, 7.5, 5.5 to 8.5, and 6.5 to 9, between appropriate integers that fall within the range. In addition, for example, the range of “10% to 30%” encompasses all integers, including numbers such as 10%, 11%, 12% and 13% as well as 30%, and any sub-ranges, such as ranges of 10% to 15%, 12% to 18%, or 20% to 30%, as well as any numbers, such as 10.5%, 15.5% and 25.5%, between appropriate integers that fall within the range.

Hereinafter, the present invention will be described in detail.

Upregulation of oncogenic miRNA-21 (miR-21) plays a pivotal role in the proliferation, migration and invasion of cancer cells. In addition to cancer cells, tumor-associated macrophages (TAMs) are enriched in miR-21 and accelerate the malignant progression of tumors in the late stages of carcinogenesis. Despite the oncogenic function of miR-21 in tumor-associated macrophages (TAM) and cancer cells, a reliable therapeutic strategy that inhibits miR-21 activity in both types of cells has not yet been developed.

Accordingly, as a result of research on an agent capable of simultaneously inhibiting the activity of miR-21 in the TAM and cancer cells, the present inventors designed a dual-target drug delivery system for miR-21 inhibitors capable of binding to both tumor cells and macrophages overexpressing the PD-L1 receptor. This peptide oligonucleotide conjugate (Pep-21) includes a PDL1-binding peptide and an anti-miR-21 inhibitor covalently linked through click chemistry. Pep-21 is preferentially internalized in both cell types, causing depletion of endogenous miR-21. According to the present invention, it was found that treatment with Pep-21 reduces migration of tumor cells, reprograms immunosuppressive M2-type TAMs into M1-type macrophages, and inhibits tumor progression. In conclusion, neutralization of miR-21 activity in both cancer cells and TAMs may be a promising strategy for an effective antitumor response.

The present inventors designed and provided a peptide-oligonucleotide conjugate in which anti-miR-21 is directly bonded to a PD-L1-binding peptide. The interaction between the peptide-oligonucleotide conjugate and the receptor thereof (PD-L1) improved the intracellular delivery potential of miRNA inhibitors. In particular, bioconjugation with PD-L1-binding peptides facilitated the target delivery of miR-21 inhibitors to specific cells within the TME, particularly macrophages and tumors overexpressing PD-L1 on the surface thereof. This interaction between PD-L1 and the PD-L1-binding peptide led to subsequent internalization through receptor-mediated endocytosis. Recently, peptides that selectively bind to PD-L1 to activate T-cell activity and inhibit tumor progression were successfully identified using a mirror-image phage display approach. The present invention provides a conjugate peptide designed through one-step copper-free click chemistry of a peptide-oligonucleotide conjugate (Pep-21) including a diarylcyclooctyne (DBCO)-functionalized anti-miR-21 inhibitor that covalently binds to the N-terminal azide residue of PDL1.

The present inventors found that Pep-21 internalizes the highly negatively charged anti-miR-21 inhibitor into cells, thereby silencing the target miR-21 in both B16 melanoma cells and M2 type macrophages (M20 through sequence-specific complementarity. Delivery of anti-miR-21 also modulated expression of miR-21-related proteins, thus increasing tumor cell death and tumor-clearing polarization of M2-type TAMs. Dual targeting of tumor cells and TAMs by Pep-21 has been shown to have the potential to significantly control tumor growth in B16 tumor-bearing mice, thus providing a promising approach for more precise and effective cancer therapies. The mechanism of action of the conjugate of the present invention on tumor cells and tumor-associated macrophages is shown in FIG. 17.

In addition, the present inventors provide anti-miRNA 155, anti-miRNA 19, anti-miRNA 17-92, anti-miRNA 128, anti-miRNA 125b, anti-miRNA 504, anti-miRNA 25 and anti-miRNA 30d miRNA inhibitors.

In addition, the present inventors provide a miRNA carrier including at least one of miRNA 34a, miRNA 194, miRNA 192, miRNA 29, miRNA 215, miRNA 200, miRNA 605, miRNA 122 and miRNA 143/145.

Meanwhile, the present invention provides the following sequences:

Sequence List 1
PD-L1-binding peptide:
NYSKPTDRQYHF
Sequence List 2
Anti-miR-21:
TCA ACA TCA GTC TGA TAA GCT A
Sequence List 3
Modified anti-miR-21:
5′-
T(PS)C_{L}(PS)A(PS)AC_{L}ATC_{L}AGT_{L}CTG_{L}ATA_{L}AG(PS)C_{L}(PS)T(PS)
A-3′
Sequence List 4
Anti-miR-155:
5′ AACCCUAUCACGAUUAGCAUUAA 3′
Sequence List 5
Anti-miR-19a:
5′-UGUAGUGCAACUAUGCAAAACU-3′
Sequence List 6
Anti-miR-19b:
5′ UGAAAUGCAAACCUGCAAAACU 3′
Sequence List 7
Anti-miR-17:
5′ CUACCUGCACUGUAAGCACUUUG 3′
Sequence List 8
Anti-miR-125b:
5′ UCACAAGUUAGGGUCUCAGGGA 3′
Sequence List 9
Anti-miR-504:
5′ GAUAGAGUGCAGACCAGGGUCU 3′
Sequence List 10
Anti-miR-25:
5′ CAAUUGCCCAAGUCUCCGCCU 3′
Sequence List 11
Anti-miR-30d:
5′ CUUCCAGUCGGGGAUGUUUACA 3′

Hereinafter, various aspects of the present invention will be described.

In the peptide-oligonucleotide conjugate according to one aspect of the present invention, the peptide is a PD-L1-binding peptide, and the oligonucleotide is an oligonucleotide having an anticancer effect or a modified oligonucleotide thereof.

In one aspect of the present invention, the PD-L1-binding peptide has the sequence of SEQ ID NO: 1.

In one aspect of the present invention, the PD-L1-binding peptide may be a PD-L1-binding peptide functionalized with an azide group.

In one aspect of the present invention, the oligonucleotide may be small interfering RNA (siRNA), small hairpin RNA (shRNA), miRNA, an antisense oligonucleotide or a nucleic acid aptamer.

In one aspect of the present invention, the oligonucleotide may be a miRNA inhibitor including at least one of anti-miRNA 21, anti-miRNA 155, anti-miRNA 19, anti-miRNA 17-92, anti-miRNA 128, anti-miRNA 125b, anti-miRNA 504, anti-miRNA 25, and anti-miRNA 30d.

In an embodiment, the miRNA inhibitor may include at least one of anti-miRNA 21, anti-miRNA 155, anti-miRNA 19, anti-miRNA 17-92, and anti-miRNA 128.

In one aspect of the present invention, the oligonucleotide may be a miRNA carrier including at least one of miRNA 34a, miRNA 194, miRNA 192, miRNA 29, miRNA 215, miRNA 200, miRNA 605, miRNA 122 and miRNA 143/145.

In an embodiment, the miRNA carrier may include at least one of miRNA 34a, miRNA 194, miRNA 192, and miRNA 29.

In one aspect of the present invention, the modified oligonucleotide thereof may include at least one of a modified internucleoside linkage, a modified sugar, and a modified nucleobase.

In an embodiment, the modified internucleoside linkage may include at least one of a phosphorothioate (PS) internucleoside linkage, a phosphodiester internucleoside linkage, a phosphotriester internucleoside linkage, a morpholino internucleoside linkage, and a protein nucleic acid (PNA) internucleoside linkage, and the modified sugar may include at least one of 2′-O-methoxyethyl, 2′-O-hydroxymethyl, 2′-hydroxyl, 2′-fluoro, and 2′,4′-LNA.

In an embodiment, the modified internucleoside linkage may be a phosphorothioate (PS) internucleoside linkage, and the modified sugar may be 2′,4′-LNA.

In one aspect of the present invention, the oligonucleotide may be anti-miRNA-21.

In one aspect of the present invention, the anti-miRNA-21 may have a sequence of SEQ ID NO: 2.

In one aspect of the present invention, the oligonucleotide may be modified anti-miRNA-21, the modified anti-miRNA-21 may include 1 to 10 phosphorothioate (PS) internucleoside linkages in an entire sequence of the oligonucleotide, and the modified anti-miRNA-21 may include 1 to 10 modified sugars of 2′,4′-LNA in the entire sequence of the oligonucleotide.

In an embodiment, the modified anti-miRNA-21 is represented by the following sequence structural formula, wherein PS represents a phosphorothioate (PS) internucleoside linkage, and the subscript L represents a modified sugar of 2′,4′-LNA.

5′-T(PS)C_{L}(PS)A(PS)AC_{L}ATC_{L}AGT_{L}CTG_{L}ATA_{L}AG(PS)C_{L}(PS)T
(PS)A-3′

In an embodiment, the sequence structural formula may be represented by a sequence of SEQ ID NO: 3.

In one aspect of the present invention, the oligonucleotide may be an oligonucleotide functionalized with diarylcyclooctyne (DBCO).

In one aspect of the present invention, the peptide-oligonucleotide conjugate may be a conjugate in which the PD-L1-binding peptide functionalized with the azide group is bound to the oligonucleotide functionalized with diarylcyclooctyne (DBCO) by a click reaction.

In one aspect of the present invention, the peptide-oligonucleotide conjugate may have a chemical structure represented by the following Formula 1:

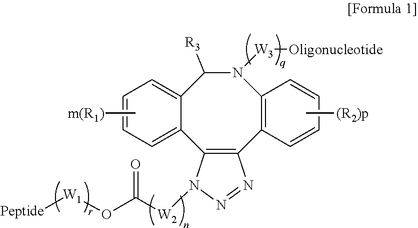

wherein
R₁ to R₃ are each independently hydrogen, a C₁-C₁₃ alkyl group, a C₁-C₆ alkoxy group, a C₆-C₁₀ aryl group, a C₃-C₁₀ cyclyl group, a C₃-C₁₀ heteroaryl group, a C₃-C₁₀ heterocyclyl group, —C(O)—(C₁-C₁₃ alkyl), —C(O)—(C₆-C₁₀ aryl), or —C(O)—(C₃-C₁₀ heteroaryl);
m, n, p, q or r each independently represents 0, 1, 2, 3 or 4;
W₁ to W₃ each independently represent —NR₃—, —NR₃CH₂—, —NR₃—C(O)—, —NR₃—C(O)—NR₄—, —NR₃—C(S)—NR₄—, —C(O)—, —C(O)CH₂—, —C(O)O—, —C(O)NR₃—, —(CH₂)—, —(CR₃R₄)—, —(CH₂)(CR₃R₄)—, —S(O)₂—, —NR₃S(O)₂—, or —S(O)₂NR₃—;
the C₁-C₁₃ alkyl group, the C₃-C₁₀ cyclyl group, and the C₁-C₆ alkoxy group include at least one substituent selected from the group consisting of hydrogen, a hydroxyl group, a halogen group, a C₁-C₁₃ alkyl group, a C₁-C₆ alkoxy group, an amino group (—NR₃R₄), a nitro group (—N(O)₂), an amide group (—(C═O)NR₃R₄), a carboxylic acid group (—C(O)OH), a nitrile group (—CN), a urea group (—NR₃(C═O)NR₄—), a sulfonamide group (—NHS(O)₂—), a sulfide group (—S—), a sulfone group (—S(O)₂—), a phosphoryl group (—P(O)R₃R₄), a C₆-C₁₀ aryl group, a C₃-C₁₀ heteroaryl group, and a C₃-C₁₀ heterocyclyl group,
the C₆-C₁₀ aryl group, the C₃-C₁₀ heteroaryl group or the C₃-C₁₀ heterocyclyl group includes at least one substituent selected from the group consisting of hydrogen, a hydroxyl group, a halogen group, a carbonyl group (—(C═O)R₃R₄), a C₁-C₃ alkyl group unsubstituted or substituted with a halogen or C₃-C₁₀ heterocyclyl group, a C₁-C₃ alkoxy group unsubstituted or substituted with a halogen or C₃-C₁₀ heterocyclyl group, C₆-C₁₀ phenoxy, an amino group (—NR₃R₄), a nitro group (—N(O)₂), an amide group (—(C═O)NR₃R₄), a carboxylic acid group (—C(O)OH), a nitrile group (—CN), a urea group (—NR₃(C═O)NR₄—), a sulfonamide group (—NHS(O)₂—), a sulfide group (—S—), a sulfone group (—S(O)₂—), a phosphoryl group (—P(O)R₃R₄), a C₆-C₁₀ aryl group, a C₃-C₁₀ heteroaryl group, and a C₃-C₁₀ heterocyclyl group; and
R₃ and R₄ are each independently hydrogen, a C₁-C₆ alkyl group, a C₁-C₆ alkenyl group, a C₁-C₆ alkynyl group, a C₆-C₁₀ aryl group, a C₃-C₁₀ heteroaryl group, or a C₃-C₁₀ heterocyclyl group, or R₃ optionally includes at least one of N, O, S, NH, C═N, C═O, —NHC(O)—, —NHC(O)NH—, —NHS(O)₂—, or SO₂ along with a nitrogen or carbon atom bound to R₄, and forms a 3- to 7-membered saturated ring that is optionally substituted with at least one of hydrogen, a C₁-C₁₃ alkyl group, a C₆-C₁₀ aryl group, a C₃-C₁₀ heteroaryl group, a hydroxyl group, a halide group, and a cyano group, and the C₃-C₁₀ heteroaryl group and the C₃-C₁₀ heterocyclyl group include at least one heteroatom selected from the group consisting of N, O, and S.

In an embodiment,
R₁ or R₂ each independently represents hydrogen, or a C₁-C₁₃ alkyl group; m or p each independently represents 0, 1, 2, 3 or 4;
R₃ is hydrogen or a C₁-C₃ alkyl group;
W₁ is —(CH₂)—, —(CR₃R₄)—, or —(CH₂)(CR₃R₄)—; r is 0, 1 or 2;
W₂ is —(CH₂)—, or —(CR₃R₄)—; n is 1 or 2;
W₃ is —(CH₂)—, or —(CR₃R₄)—; and q is 0, 1 or 2.

In one aspect of the present invention, in Formula 1, the peptide may be a PD-L1-binding peptide, and the PD-L1-binding peptide may have a sequence of SEQ ID NO: 1, and in Formula 1, the oligonucleotide may be anti-miRNA-21 or modified anti-miRNA-21, the anti-miRNA-21 may have a sequence of SEQ ID NO: 2, and the modified anti-miRNA-21 may have a sequence of SEQ ID NO: 3.

In one aspect of the present invention, the peptide-oligonucleotide conjugate may inhibit expression of at least one of miR-21, miRNA 155, miRNA 19, miRNA 17-92, miRNA 128, miRNA 125b, miRNA 504, miRNA 25, and miRNA 30d in cells.

In another aspect of the present invention, the peptide-oligonucleotide conjugate may improve expression of at least one of miRNA 34a, miRNA 194, miRNA 192, miRNA 29, miRNA 215, miRNA 200, miRNA 605, miRNA 122, and miRNA 143/145 in cells.

In another aspect, the present invention provides a pharmaceutical formulation containing the peptide-oligonucleotide conjugate according to one aspect of the present invention.

In another aspect, the oligonucleotide may be any one of the following: anti-miRNA 155 represented by SEQ ID NO: 4; anti-miRNA 19a represented by SEQ ID NO: 5; anti-miRNA 19b represented by SEQ ID NO: 6; anti-miRNA 17 represented by SEQ ID NO: 7; anti-miRNA 125b represented by SEQ ID NO: 8; anti-miRNA 504 represented by SEQ ID NO: 9; anti-miRNA 25 represented by SEQ ID NO: 10; and anti-miRNA 30d represented by SEQ ID NO: 11.

In another aspect, the present invention provides a pharmaceutical formulation for preventing, ameliorating or treating cancer containing the peptide-oligonucleotide conjugate according to one aspect of the present invention.

In another aspect of the present invention, the cancer may be melanoma, breast cancer, lung cancer, colon cancer, ovarian cancer, head and neck cancer, or neuroblastoma.

In another aspect, the present invention provides a method for preparing the peptide-oligonucleotide conjugate according to one aspect of the present invention, the method including (a) functionalizing PD-L1-binding peptide with an azide group, (b) functionalizing oligonucleotide with diarylcyclooctyne (DBCO), and (c) reacting oligonucleotide functionalized with diarylcyclooctyne (DBCO) with the PD-L1-binding peptide functionalized with the azide group by a click reaction to prepare a peptide-oligonucleotide conjugate.

In another aspect of the present invention, the PD-L1-binding peptide functionalized with the azide group may be a compound represented by the following Formula 2, and the oligonucleotide functionalized with diarylcyclooctyne (DBCO) may be a compound represented by the following Formula 3:

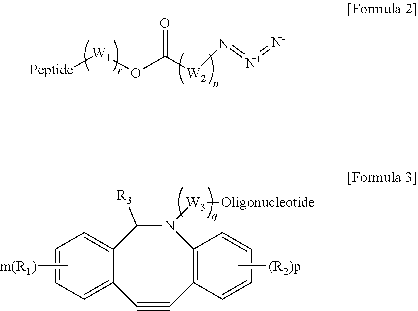

wherein
R₁ to R₃ each independently represent hydrogen, a C₁-C₁₃ alkyl group, a C₁-C₆ alkoxy group, a C₆-C₁₀ aryl group, a C₃-C₁₀ cyclyl group, a C₃-C₁₀ heteroaryl group, a C₃-C₁₀ heterocyclyl group, —C(O)—(C₁-C₁₃ alkyl), —C(O)—(C₆-C₁₀ aryl), or —C(O)—(C₃-C₁₀ heteroaryl);
m, n, p, q or r each independently represents 0, 1, 2, 3 or 4;
W₁ to W₃ each independently represent —NR₃—, —NR₃CH₂—, —NR₃—C(O)—, —NR₃—C(O)—NR₄—, —NR₃—C(S)—NR₄—, —C(O)—, —C(O)CH₂—, —C(O)O—, —C(O)NR₃—, —(CH₂)—, —(CR₃R₄)—, —(CH₂)(CR₃R₄)—, —S(O)₂—, —NR₃S(O)₂—, or —S(O)₂NR₃;
wherein the C₁-C₁₃ alkyl group, the C₃-C₁₀ cyclyl group, and the C₁-C₆ alkoxy group include at least one substituent selected from the group consisting of hydrogen, a hydroxyl group, a halogen group, a C₁-C₁₃ alkyl group, a C₁-C₆ alkoxy group, an amino group (—NR₃R₄), a nitro group (—N(O)₂), an amide group (—(C═O)NR₃R₄), a carboxylic acid group (—C(O)OH), a nitrile group (—CN), a urea group (—NR₃(C═O)NR₄—), a sulfonamide group (—NHS(O)₂—), a sulfide group (—S—), a sulfone group (—S(O)₂—), a phosphoryl group (—P(O)R₃R₄), a C₆-C₁₀ aryl group, a C₃-C₁₀ heteroaryl group, and a C₃-C₁₀ heterocyclyl group,
the C₆-C₁₀ aryl group, the C₃-C₁₀ heteroaryl group or the C₃-C₁₀ heterocyclyl group includes at least one substituent selected from the group consisting of hydrogen, a hydroxyl group, a halogen group, a carbonyl group (—(C═O)R₃R₄), a C₁-C₃ alkyl group unsubstituted or substituted with a halogen or C₃-C₁₀ heterocyclyl group, a C₁-C₃ alkoxy group unsubstituted or substituted with a halogen or C₃-C₁₀ heterocyclyl group, C₆-C₁₀ phenoxy, an amino group (—NR₃R₄), a nitro group (—N(O)₂), an amide group (—(C═O)NR₃R₄), a carboxylic acid group (—C(O)OH), a nitrile group (—CN), a urea group (—NR₃(C═O)NR₄—), a sulfonamide group (—NHS(O)₂—), a sulfide group (—S—), a sulfone group (—S(O)₂—), a phosphoryl group (—P(O)R₃R₄), a C₆-C₁₀ aryl group, a C₃-C₁₀ heteroaryl group, and a C₃-C₁₀ heterocyclyl group; and
R₃ and R₄ each independently represent hydrogen, a C₁-C₆ alkyl group, a C₁-C₆ alkenyl group, a C₁-C₆ alkynyl group, a C₆-C₁₀ aryl group, a C₃-C₁₀ heteroaryl group, or a C₃-C₁₀ heterocyclyl group, or R₃ optionally includes at least one of N, O, S, NH, C═N, C═O, —NHC(O)—, —NHC(O)NH—, —NHS(O)₂—, or SO₂ along with a nitrogen or carbon atom bound to R₄, and forms a 3- to 7-membered saturated ring that is optionally substituted with at least one of hydrogen, a C₁-C₁₃ alkyl group, a C₆-C₁₀ aryl group, a C₃-C₁₀ heteroaryl group, a hydroxyl group, a halide group, and a cyano group, and the C₃-C₁₀ heteroaryl group and the C₃-C₁₀ heterocyclyl group include at least one heteroatom selected from the group consisting of N, O, and S.

In an embodiment, R₁ or R₂ may each independently represent hydrogen, or a C₁-C₁₃ alkyl group; m or p each independently represents 0, 1, 2, 3 or 4; R₃ is hydrogen, or a C₁-C₃ alkyl group; W₁ is —(CH₂)—, —(CR₃R₄)—, or —(CH₂)(CR₃R₄)—; r is 0, 1 or 2; W₂ is —(CH₂)—, or —(CR₃R₄)—; n is 1 or 2; W₃ is —(CH₂)—, or —(CR₃R₄)—; and q is 0, 1 or 2.

In another aspect of the present invention, the click reaction in step (c) may be performed in a pH 6.5-8.0 buffer solution at 700 rpm to 1,500 rpm and a temperature of 30° C. to 45° C.

In another aspect of the present invention, in step (c), the oligonucleotide functionalized with diarylcyclooctyne (DBCO) and the PD-L1-binding peptide functionalized with the azide group may be synthesized at a molar ratio of 1:1.5 to 1:2.5.

In another aspect of the present invention, in step (C), the reaction may be performed for 1 to 3 hours.

In another aspect of the present invention, the PD-L1-binding peptide may have a sequence of SEQ ID NO: 1.

In another aspect of the present invention, the anti-miRNA-21 may have a sequence of SEQ ID NO: 2 or SEQ ID NO: 3.

### MODE FOR INVENTION

Hereinafter, the present invention will be described in more detail with reference to specific Examples. However, the following Examples are provided only for better understanding of the present invention, and should not be construed as limiting the scope of the present invention.

### Example 1: Synthesis of Peptide-Oligonucleotide Composite (Pep-21)

PDL1-binding peptides modified with N-terminal azide were obtained from Peptron Company (Daejeon, Korea). The specific sequence of the PDL1-binding peptide modified with azide is as follows.

Asn-Tyr-Ser-Lys-Pro-Thr-Asp-Arg-Gln-Tyr-His-Phe
(N3-NYSKPTDRQYHF, d-form)

5′-Diarylcyclooctyne (DBC0)-modified-anti-miR-21, Cy5-labeled DBCO-21, constituted with DNA (DBCO-21, sequence: DBCO-5′-TCA ACA TCA GTC TGA TAA GCT A-3′), control miR-21 DNA (sequence: 5′-UAG CUU AUC AGA CUG AUG UUG A-3′), and negative control miRNA were obtained from Bioneer (Daejeon, Republic of Korea).

Azide-functionalized PDL1-binding peptide (3.3 μg, 2 nmole) and DBCO-functionalized Anti-miR-21 (7.8 μg, 1 nmole) were synthesized by non-copper click reaction in PBS buffer (10 μl, pH 7.4) at 37° C. and 1,100 rpm. After incubation, the resulting PDL1-binding peptide-anti-miR-21 conjugate (Pep-21) was identified on an agarose gel and then identified in Example 4 below by MALDI-TOF mass spectrometry.

### Example 2. Comparison of Synthesis Efficiency of Pep-21

The synthesis efficiency of the PDL1-binding peptide and DBCO-anti-21 at various molar ratios for various synthesis periods were compared. All experiments were performed in PBS (pH 7.4) at 37° C.

### (1) Comparison of Molar Ratio

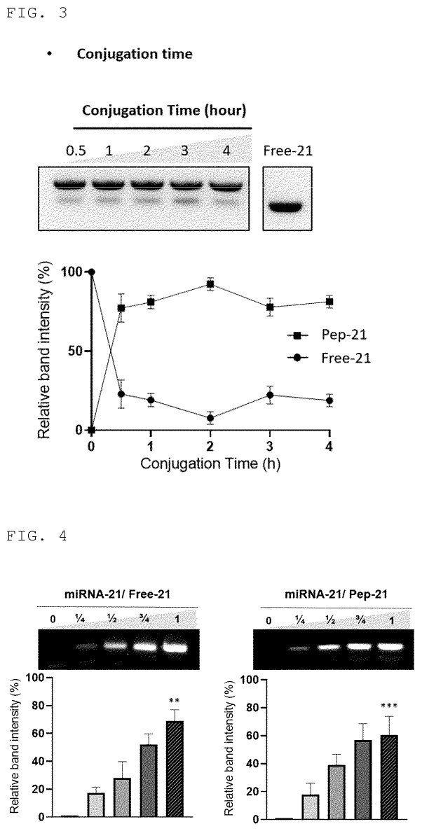

The peptide, DBCO-anti-21 was synthesized in various molar ratios (0.5, 1, 2, 4, 8) for 2 hours. The test results are shown in FIG. 2.

### (2) Comparison in Conjugation Time

Peptide and DBCO-anti-21 were synthesized at a ratio of 2:1 for 0.5, 1, 2, 3, or 4 hours.

The test results are shown in FIG. 3.

The result of agarose gel electrophoresis showed that when the molar ratio of PDL1-binding peptide to DBCO-Anti-21 is 2:1, the most optimal synthesis efficiency was obtained.

Synthesis was performed at a molar ratio of 2:1 for various periods of time (for 0.5, 1, 2, 3, and 4 hours). At this time, agarose gel electrophoresis showed that when the synthesis time was 2 hours, the most optimal synthesis efficiency was obtained.

### Example 3. Characterization of Pep-21 (PDL1-Binding Peptide-Anti-miR-21 Conjugate)

The binding affinity of miRNA-21 and Pep-21 was evaluated through agarose gel electrophoresis, compared to Free-21.

Synthetic Pep-21 (10 pmole) was reacted with miRNA-21 and negative control miRNA in 100 μL of PBS under the same conditions as above, in other words, at 37° C. for 1 hour and then whether or not pep-21 recognizes and binds to a specific sequence of miRNA-21 was identified by agarose gel electrophoresis.

(1) Confirmation-1 of Binding Affinity Between miRNA-21 and Pep-21

The binding affinity between miRNA-21 and Pep-21 was determined, compared to Free-21.

It was confirmed that when Free-21 (10 pmole) was annealed with miR-21 (0, 0.25, 0.5, 0.75 and 1) in 100 μL of PBS at 37° C. for 1 hour, the binding affinity increased as the concentration increased.

It was confirmed that when synthetic Pep-21 (10 pmole) was annealed with miR-21 (0, 0.25, 0.5, 0.75 and 1) in 100 μL of PBS at 37° C. for 1 hour, the binding affinity increased as the concentration increased.

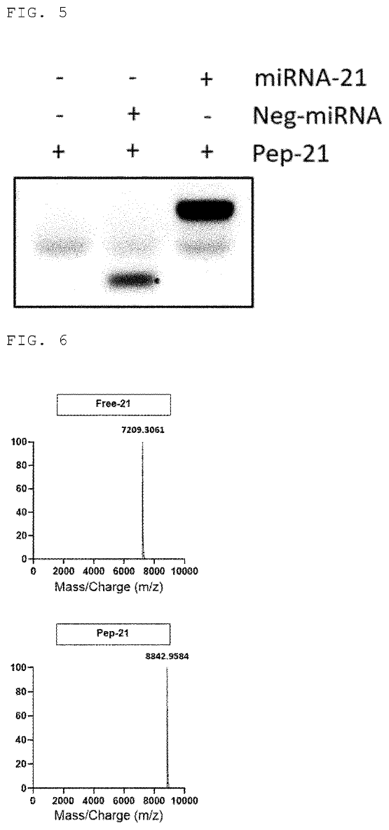

The test results are shown in FIG. 4.

(2) Confirmation-2 of Binding Affinity Between miRNA-21 and Pep-21

Whether or not Pep-21 recognizes and binds to a specific sequence to miRNA-21 was determined using the negative control miRNA.

The test results are shown in FIG. 5.

Upon reaction with Neg-miRNA, no band shift was observed.

Upon reaction with miRNA-21, a band shift was observed and thus a synthetic form could be determined based thereon.

That is, it was confirmed that Pep-21 recognizes a specific sequence and selectively binds to miRNA-21.

### Example 4. Characterization of Pep-21 (PDL1-Binding Peptide-Anti-miR-21 Conjugates)

Molecular weight analysis was performed using a Maldi-Tof mass spectrometer.

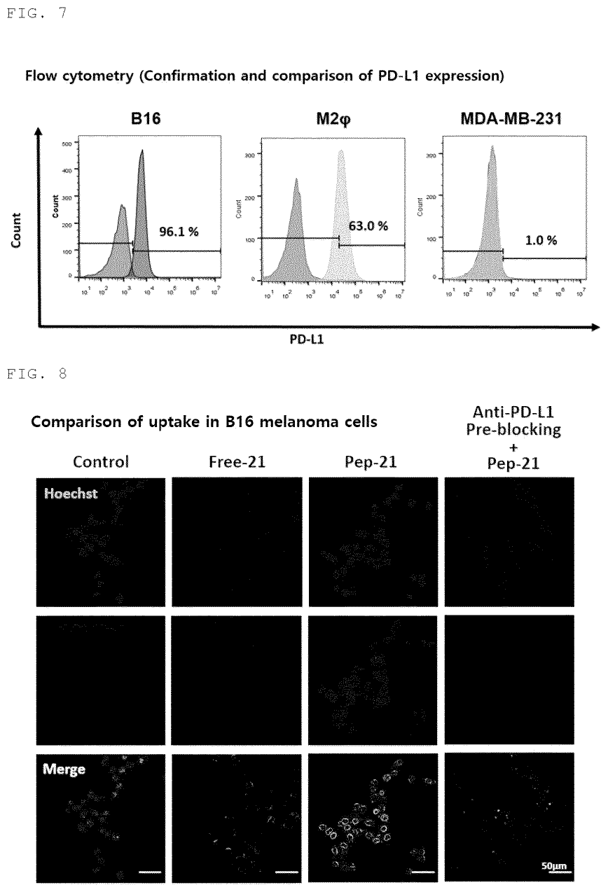

The test results are shown in FIG. 6.

The molecular weight of Free-21 was about 7209.3061 g/mol and the molecular weight of Pep-21 was about 8842.9584 g/mol. Synthesis of pep-21 was identified by the increase in molecular weight.

### Example 5. Confirmation of Amounts of PD-L1 Expressed (B16, M2ϕ, MDA-MB-231)

Three cell lines were used for comparative analysis of cellular uptake in this research. The levels of PD-L1 expressed in each of the cell lines B16 (melanoma model), M2ϕ (M2 bone marrow-derived macrophages), and MDA-MB-231 (human breast cancer cells) were determined by flow cytometry.

The test was performed by staining PD-L1 expressed on the surface of the cells with an APC-fluorescent PD-L1 antibody to measure the expression thereof in each cell line.

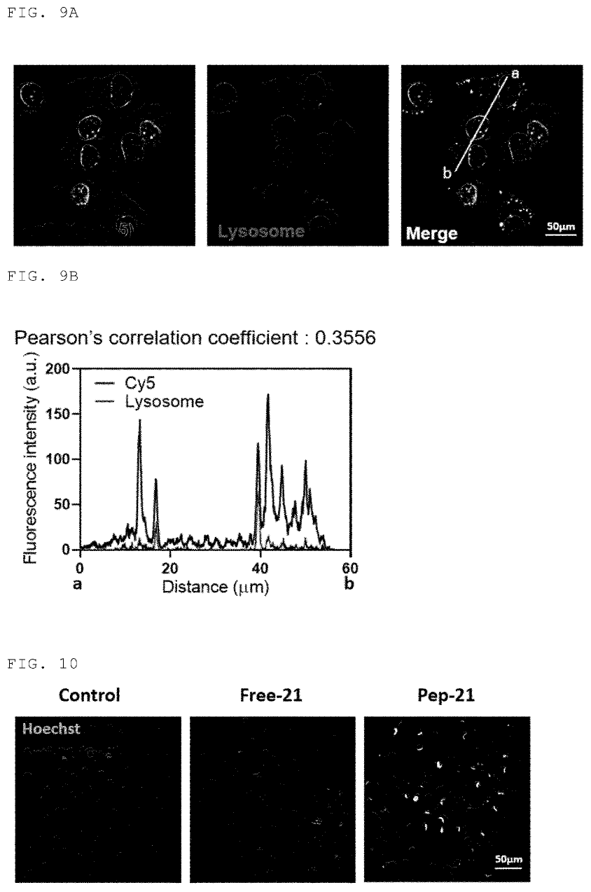

The test results are shown in FIG. 7.

It was confirmed that PD-L1 was overexpressed in B16 and M2ϕ cell lines, whereas PD-L1 was relatively not expressed in MDA-MB-231.

### Example 6-1. Comparison of Uptake of Pep-21 in B16 Melanoma Cells

### (1) Comparison of Uptake in B16 Melanoma Cells

Melanoma cells (B16) were treated with 300 nM miRNA-21 labeled with Cy5 fluorescent dye for 6 hours and the degree of intracellular uptake was then observed with a fluorescence microscope.

The test results are shown in FIG. 8.

The treatment with Pep-21, compared to free-21, which was observed in very small amounts in cells, was greatly advantageous in intracellular delivery of anti-miR21.

When blocking with PD-L1 antibody under the same conditions, cellular uptake decreased. This indicates that Pep-21 was internalized by PD-L1-receptor-mediated endocytosis.

### Example 6-2. Co-Localization of Pep-21 and Lysosome in B16 Melanoma Cells

Melanoma cells (B16) were treated with 300 nM miRNA-21 labeled with a Cy5 fluorescence dye and lysosomes were stained with 1 μM LysoTracker™ Green DND-26 1 hour before fluorescence imaging.

Then, the co-localization of Pep-21 absorbed into cells and lysosomes was confirmed with a fluorescence microscope.

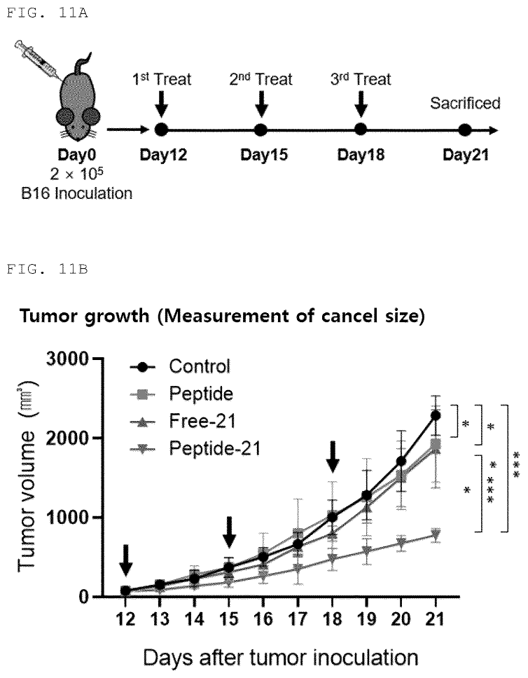

The test results are shown in FIGS. 9A and 9B.

The correlation coefficient between Pep-21 and lysosome was 0.3556, which indicates that Pep-21 successfully escapes endosomes and releases anti-miR21 into the cytoplasm.

### Example 7. Comparison of Uptake in M2ϕ (M2 Bone Marrow-Derived Macrophages)

miRNA-21 was treated with 300 nM miRNA-21 labeled with Cy5 fluorescent dye in M2ϕ (M2 bone marrow-derived macrophages) for 6 hours and the degree of uptake into cells was observed with a fluorescence microscope.

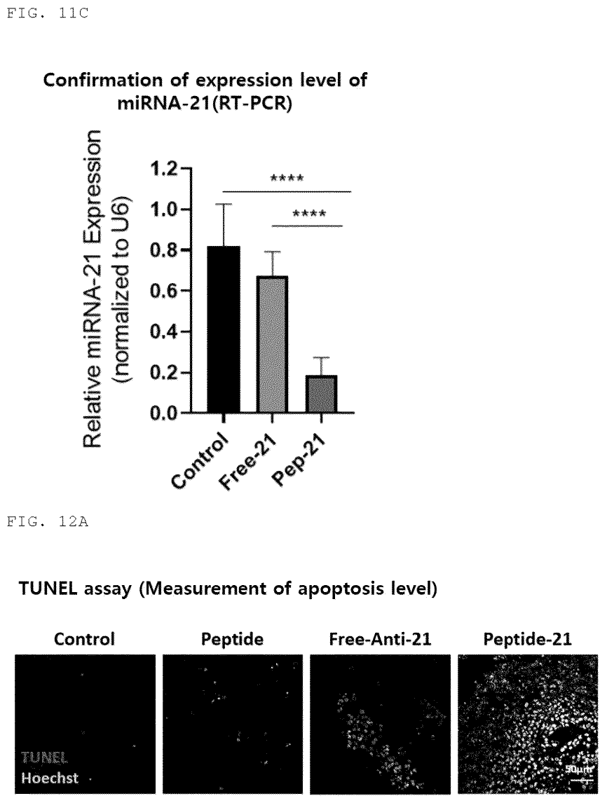

The test results are shown in FIG. 10.

Compared to Free-21, Pep-21 successfully delivered anti-21 to the cytoplasm.

### Example 8. Tests of Anti-Tumor Effect in Animals

Treatment of a tumor animal model (B16 melanoma) with 500 pmole of Pep-21 3 times at 3-day intervals was found to inhibit cancer growth.

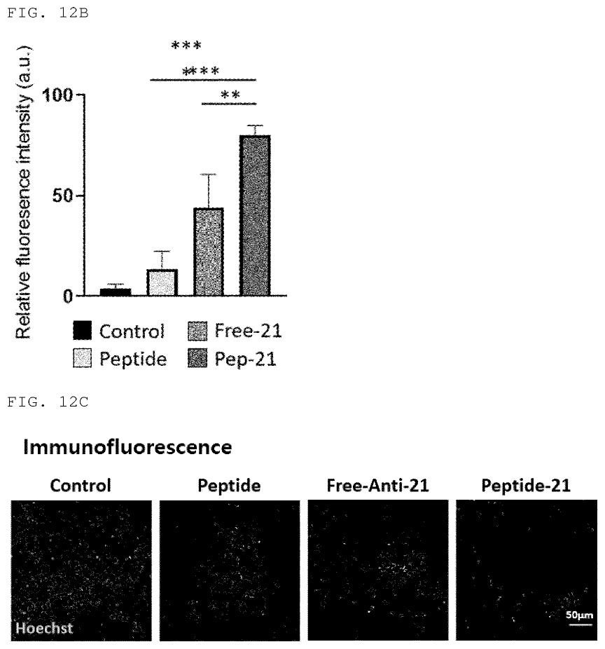

On day 12 after tumor inoculation (tumor size of about 70 mm³), PBS, peptide, Free-21, and Pep-21 were intratumorally injected three times at intervals of three days. The test outline is shown in FIG. 11A.

The test results are shown in FIGS. 11B and 11C.

The result of analysis of the amount of miRNA-21 expressed in cancer tissue treated with Pep-21 by real-time polymerase chain reaction (RT-PCR) showed that the amount of miRNA-21 that was expressed was reduced compared to the control group.

### Example 9. Confirmation of Apoptosis and Angiogenesis in Pep-21-Treated Tumor Tissue

21 days after the animal test, the excised tumor tissue was analyzed by immunofluorescence.

TUNEL assay was performed on tumor tissue to determine the degree of apoptosis.

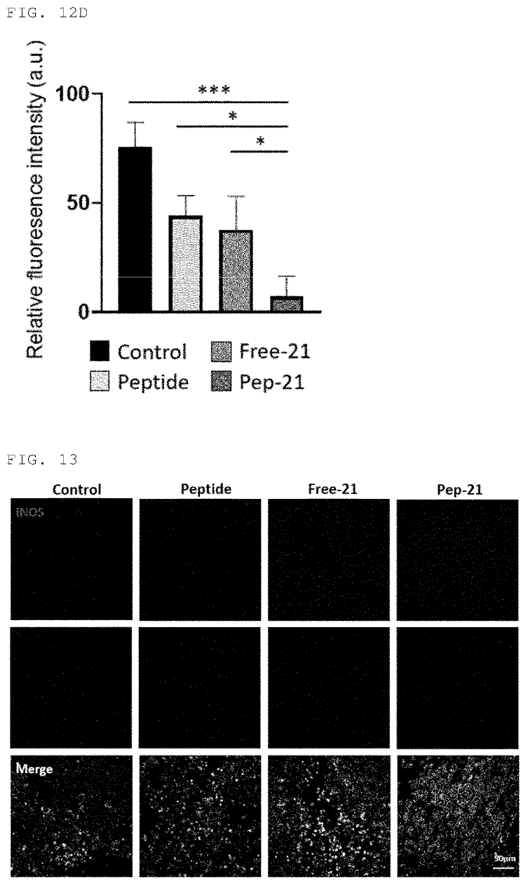

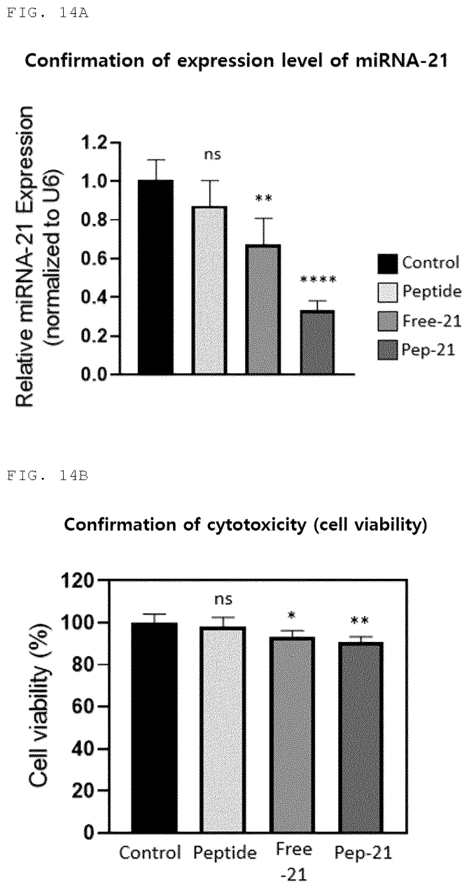

The test results are shown in FIGS. 12A and 12B. The results showed that the degree of apoptosis in the Pep-21 group increased compared to the control group.

In addition, immunofluorescence was performed using CD31, an angiogenesis marker.

The test results are shown in FIGS. 12C and 12D. The results showed that the expression of the Pep-21 group decreased compared to the control group.

### Example 10. Analysis of Macrophages in Tumor Tissues Treated with Pep-21

21 days after the animal test, tumor-associated macrophages infiltrating the excised tumor tissue were analyzed by immunofluorescence (IF).

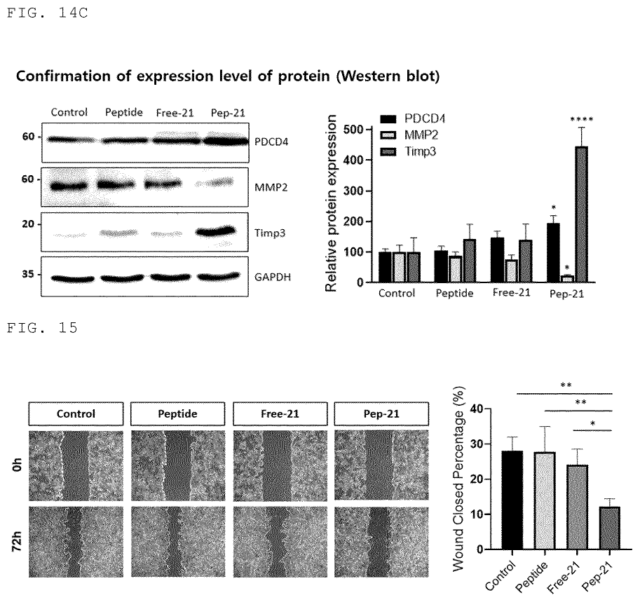

The test results are shown in FIG. 13.

The result of IF showed that iNOS (M1 marker) increased and Arginasel (M2 marker) decreased in tumor tissue treated with Pep-21.

### Example 11. Confirmation of Anti-Tumor Effect of Pep-21 in B16 Melanoma Cells

(1) Real-time polymerase chain reaction (RT-PCR) was performed to determine whether or not miRNA-21 expression was inhibited by treatment with Pep-21. B16 cells were treated with 300 nM of Pep-21 for 18 hours, RNA was isolated, cDNA was synthesized, and then real-time polymerase chain reaction (RT-PCR) was performed using miRNA-21 primer.

The test results are shown in FIG. 14A. The result showed that treatment of the B16 cell line with Pep-21 decreased the expression of miRNA-21.

(2) To determine the cytotoxicity of this substance, B16 cells were treated with 300 nM Pep-21 for 24 hours and the cytotoxicity was confirmed using cell counting Kit-8 (CCK-8).

The test results are shown in FIG. 14B. The result showed that when the B16 cell line was treated with Pep-21, there was almost no cytotoxicity.

(3) Confirmation of protein expression level

B16 cells were treated with 300 nM Pep-21 in serum free media for 24 hours, protein assay was performed and western blot was performed.

miRNA-21 related markers: programmed cell death 4 (PDCD4), matrix metalloproteinase-2 (MMP2), tissue inhibitor of metalloproteinase-3 (TIMP3)

The test results are shown in FIG. 14C.

Upon treatment with Pep-21, the expression levels of PDCD4, MMP2, and Timp3, which are miRNA-21-related markers, were detected. The result showed that PDCD4 and Timp3 expressions increased, whereas MMP2 expression decreased.

### Example 12. Confirmation of Cell Migration of Pep-21 in B16 Melanoma Cells

A scratch-wound healing assay was performed in order to analyze cell migration.

B16 cells were wounded using a 200 μL pipette tip, treated with 300 nM Pep-21 in serum free media for 48 hours, and then stabilized in fresh growth media for 24 hours.

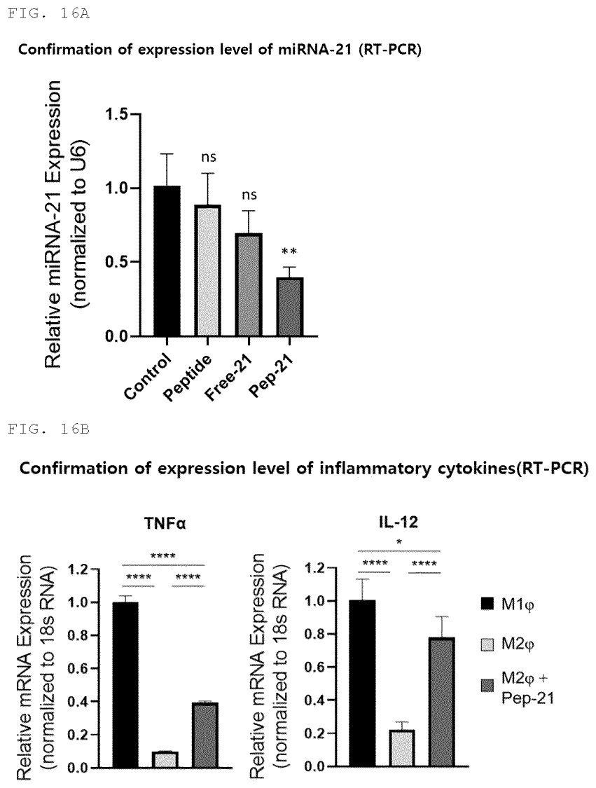

The test results are shown in FIG. 15.

72 hours after wounding, cell migration was observed. The result showed that cancer cell migration was further inhibited when treated with Pep-21 compared to the control group.

### Example 13. Confirmation of Anti-Tumor Effect of Pep-21 in M2ϕ (M2 Bone Marrow-Derived Macrophages)

Real-time polymerase chain reaction (RT-PCR) was performed to determine whether or not treatment with Pep-21 inhibited the expression of miRNA-21. M2ϕ was treated with 300 nM of Pep-21 for 18 hours, RNA was isolated and then real-time polymerase chain reaction (RT-PCR) was performed.

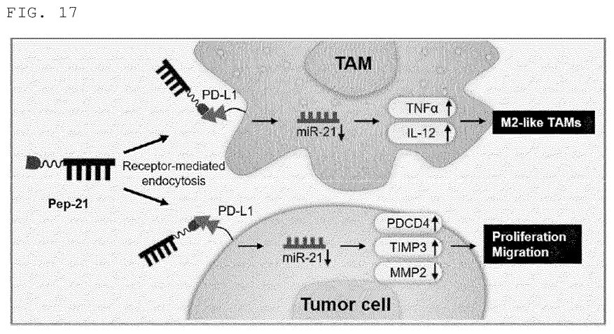

The test results are shown in FIG. 16A. Treatment of M2ϕ with Pep-21 inhibited expression of miRNA-21.

In addition, the expression level of inflammatory cytokines was determined through RT-PCR.

The test results are shown in FIG. 16B. Treatment of M2ϕ with Pep-21 promoted mRNA expression of TNF-α and IL-12, which are inflammatory cytokines. This result suggests that Pep-21 inhibits miRNA-21 expression and converts the phenotype of the macrophages from M2ϕ to M1ϕ.

Although embodiments of the present invention have been described with reference to the drawings, it will be obvious to those skilled in the art that the embodiments can be implemented in other specific forms without changing the technical concepts or essential features of the present invention. Therefore, it should be construed that the aforementioned embodiments are illustrative and not restrictive in all respects.

1. A peptide-oligonucleotide conjugate,
wherein the peptide is a PD-L1-binding peptide, and
the oligonucleotide is an oligonucleotide or a modified oligonucleotide thereof.

2. The peptide-oligonucleotide conjugate according to claim 1, wherein the PD-L1-binding peptide has a sequence of SEQ ID NO: 1.

3. The peptide-oligonucleotide conjugate according to claim 1, wherein the PD-L1-binding peptide is a PD-L1-binding peptide functionalized with an azide group.

4. The peptide-oligonucleotide conjugate according to claim 1, wherein the oligonucleotide is small interfering RNA (siRNA), small hairpin RNA (shRNA), miRNA, an antisense oligonucleotide or a nucleic acid aptamer.

5. The peptide-oligonucleotide conjugate according to claim 1, wherein the oligonucleotide is a miRNA inhibitor comprising at least one of anti-miRNA 21, anti-miRNA 155, anti-miRNA 19, anti-miRNA 17-92, anti-miRNA 128, anti-miRNA 125b, anti-miRNA 504, anti-miRNA 25, and anti-miRNA 30d.

6. The peptide-oligonucleotide conjugate according to claim 5, wherein the miRNA inhibitor comprises at least one of anti-miRNA 21, anti-miRNA 155, anti-miRNA 19, anti-miRNA 17-92, and anti-miRNA 128.

7. The peptide-oligonucleotide conjugate according to claim 1, wherein the oligonucleotide is an miRNA carrier comprising at least one of miRNA 34a, miRNA 194, miRNA 192, miRNA 29, miRNA 215, miRNA 200, miRNA 605, miRNA 122, and miRNA 143/145.

8. The peptide-oligonucleotide conjugate according to claim 7, wherein the miRNA carrier comprises at least one of miRNA 34a, miRNA 194, miRNA 192, and miRNA 29.

9. The peptide-oligonucleotide conjugate according to claim 1, wherein the modified oligonucleotide thereof comprises at least one of a modified internucleoside linkage, a modified sugar, and a modified nucleobase.

10. The peptide-oligonucleotide conjugate according to claim 9, wherein the modified internucleoside linkage comprises at least one of a phosphorothioate (PS) internucleoside linkage, a phosphodiester internucleoside linkage, a phosphotriester internucleoside linkage, a morpholino internucleoside linkage, and a protein nucleic acid (PNA) internucleoside linkage, and
the modified sugar comprises at least one of 2′-O-methoxyethyl, 2′-O-hydroxymethyl, 2′-hydroxyl, 2′-fluoro, and 2′,4′-LNA.

11. The peptide-oligonucleotide conjugate according to claim 10, wherein the modified internucleoside linkage is a phosphorothioate (PS) internucleoside linkage, and
the modified sugar is 2′,4′-LNA.

12. The peptide-oligonucleotide conjugate according to claim 1, wherein the oligonucleotide is anti-miRNA-21.

13. The peptide-oligonucleotide conjugate according to claim 1, wherein the anti-miRNA-21 has a sequence of SEQ ID NO: 2.

14. The peptide-oligonucleotide conjugate according to claim 1, wherein the oligonucleotide is modified anti-miRNA-21,
the modified anti-miRNA-21 comprises 1 to 10 phosphorothioate (PS) internucleoside linkages in an entire sequence of the oligonucleotide, and
the modified anti-miRNA-21 comprises 1 to 10 modified sugars of 2′,4′-LNA in the entire sequence of the oligonucleotide.

15. The peptide-oligonucleotide conjugate according to claim 14, wherein the modified anti-miRNA-21 is represented by the following sequence structural formula,
wherein PS represents a phosphorothioate (PS) internucleoside linkage, and the subscript L represents a modified sugar of 2′,4′-LNA,
5′-T(PS)C_{L}(PS)A(PS)AC_{L}ATC_{L}AGT_{L}CTG_{L}ATA_{L}AG(PS)C_{L}(PS)T(PS)A-3′

16. The peptide-oligonucleotide conjugate according to claim 15, wherein the sequence structural formula is represented by a sequence of SEQ ID NO: 3.

17. The peptide-oligonucleotide conjugate according to claim 1, wherein the oligonucleotide is an oligonucleotide functionalized with diarylcyclooctyne (DBCO).

18. The peptide-oligonucleotide conjugate according to claim 1, wherein the peptide-oligonucleotide conjugate is a conjugate in which the PD-L1-binding peptide functionalized with the azide group is bound to the oligonucleotide functionalized with diarylcyclooctyne (DBCO) by a click reaction.

19. The peptide-oligonucleotide conjugate according to claim 18, wherein the peptide-oligonucleotide conjugate has a chemical structure represented by the following Formula 1: 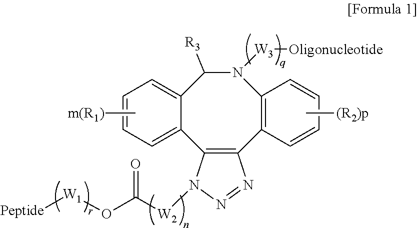
wherein R₁ to R₃ are each independently hydrogen, a C₁-C₁₃ alkyl group, a C₁-C₆ alkoxy group, a C₆-C₁₀ aryl group, a C₃-C₁₀ cyclyl group, a C₃-C₁₀ heteroaryl group, a C₃-C₁₀ heterocyclyl group, —C(O)—(C₁-C₁₃ alkyl), —C(O)—(C₆-C₁₀ aryl), or —C(O)—(C₃-C₁₀ heteroaryl);
m, n, p, q or r each independently represents 0, 1, 2, 3 or 4;
W₁ to W₃ each independently represent —NR₃—, —NR₃CH₂—, —NR₃—C(O)—, —NR₃—C(O)—NR₄—, —NR₃—C(S)₁₃ NR₄—, —C(O)—, —C(O)CH₂—, —C(O)O—, —C(O)NR₃—, —(CH₂)—, —(CR₃R₄)—, —(CH₂)(CR₃R₄)—, —S(O)₂—, —NR₃S(O)₂—, or —S(O)₂NR₃—;
the C₁-C₁₃ alkyl group, the C₃ -C₁₀ cyclyl group, and the C₁-C₆ alkoxy group comprise at least one substituent selected from the group consisting of hydrogen, a hydroxyl group, a halogen group, a C₁-C₁₃ alkyl group, a C₁-C₆ alkoxy group, an amino group (—NR₃R₄), a nitro group (—N(O)₂), an amide group (—(C═O)NR₃R₄), a carboxylic acid group (—C(O)OH), a nitrile group (—CN), a urea group (—NR₃(C═O)NR₄—), a sulfonamide group (—NHS(O)₂—), a sulfide group (—S—), a sulfone group (—S(O)₂—), a phosphoryl group (—P(O)R₃R₄), a C₆-C₁₀ aryl group, a C₃-C₁₀ heteroaryl group, and a C₃-C₁₀ heterocyclyl group,
the C₆-C₁₀ aryl group, the C₃-C₁₀ heteroaryl group or the C₃-C₁₀ heterocyclyl group comprises at least one substituent selected from the group consisting of hydrogen, a hydroxyl group, a halogen group, a carbonyl group (—(C═O)R₃R₄), a C₁-C₃ alkyl group unsubstituted or substituted with a halogen or C₃-C₁₀ heterocyclyl group, a C₁-C₃ alkoxy group unsubstituted or substituted with a halogen or C₃-C₁₀ heterocyclyl group, C₆-C₁₀ phenoxy, an amino group (—NR₃R₄), a nitro group (—N(O)₂), an amide group (—(C═O)NR₃R₄), a carboxylic acid group (—C(O)OH), a nitrile group (—CN), a urea group (—NR₃(C═O)NR₄—), a sulfonamide group (—NHS(O)₂—), a sulfide group (—S—), a sulfone group (—S(O)₂—), a phosphoryl group (—P(O)R₃R₄), a C₆-C₁₀ aryl group, a C₃-C₁₀ heteroaryl group, and a C₃-C₁₀ heterocyclyl group; and
R₃ and R₄ are each independently hydrogen, a C₁-C₆ alkyl group, a C₁-C₆ alkenyl group, a C₁-C₆ alkynyl group, a C₆-C₁₀ aryl group, a C₃-C₁₀ heteroaryl group, or a C₃-C₁₀ heterocyclyl group, or R₃ optionally comprises at least one of N, O, S, NH, C═N, C═O, —NHC(O)—, —NHC(O)NH—, —NHS(O)₂—, or SO₂ along with a nitrogen or carbon atom bound to R₄, and forms a 3- to 7-membered saturated ring that is optionally substituted with at least one of hydrogen, a C₁-C₁₃ alkyl group, a C₆-C₁₀ aryl group, a C₃-C₁₀ heteroaryl group, a hydroxyl group, a halide group, and a cyano group, and the C₃-C₁₀ heteroaryl group and the C₃-C₁₀ heterocyclyl group comprise at least one heteroatom selected from the group consisting of N, O, and S.

20. The peptide-oligonucleotide conjugate according to claim 19, wherein
R₁ or R₂ each independently represents hydrogen, or a C₁-C₁₃ alkyl group;
m or p each independently represents 0, 1, 2, 3 or 4;
R₃ is hydrogen or a C₁-C₃ alkyl group;
W₁ is —(CH₂)—, —(CR₃R₄)—, or —(CH₂)(CR₃R₄)—;
r is 0, 1 or 2;
W₂ is —(CH₂)—, or —(CR₃R₄)—;
n is 1 or 2;
W₃ is —(CH₂)—, or —(CR₃R₄)—; and
q is 0, 1 or 2.

21. The peptide-oligonucleotide conjugate according to claim 20, wherein, in Formula 1, the peptide is a PD-L1-binding peptide, and the PD-L1-binding peptide has a sequence of SEQ ID NO: 1, and in Formula 1, the oligonucleotide is anti-miRNA-21 or modified anti-miRNA-21, the anti-miRNA-21 has a sequence of SEQ ID NO: 2, and the modified anti-miRNA-21 has a sequence of SEQ ID NO: 3.

22. The peptide-oligonucleotide conjugate according to claim 1, wherein the peptide-oligonucleotide conjugate inhibits expression of at least one of miR-21, miRNA 155, miRNA 19, miRNA 17-92, miRNA 128, miRNA 125b, miRNA 504, miRNA 25, and miRNA 30d in cells.

23. The peptide-oligonucleotide conjugate according to claim 1, wherein the peptide-oligonucleotide conjugate improves expression of at least one of miRNA 34a, miRNA 194, miRNA 192, miRNA 29, miRNA 215, miRNA 200, miRNA 605, miRNA 122, and miRNA 143/145 in cells.

24. The peptide-oligonucleotide conjugate according to claim 1, wherein the oligonucleotide is any one of the following:
anti-miRNA 155 represented by SEQ ID NO: 4;
anti-miRNA 19a represented by SEQ ID NO: 5;
anti-miRNA 19b represented by SEQ ID NO: 6;
anti-miRNA 17 represented by SEQ ID NO: 7;
anti-miRNA 125b represented by SEQ ID NO: 8;
anti-miRNA 504 represented by SEQ ID NO: 9;
anti-miRNA 25 represented by SEQ ID NO: 10; and
anti-miRNA 30d represented by SEQ ID NO: 11.

25. A pharmaceutical formulation for preventing, ameliorating or treating cancer comprising the peptide-oligonucleotide conjugate according to claim 1.

26. The pharmaceutical formulation according to claim 25, wherein the cancer is melanoma, breast cancer, lung cancer, colon cancer, ovarian cancer, head and neck cancer, or neuroblastoma.

27. A method for preparing the peptide-oligonucleotide conjugate according to claim 1, the method comprising:
(a) functionalizing PD-L1-binding peptide with an azide group;
(b) functionalizing oligonucleotide with diarylcyclooctyne (DBCO); and
(c) reacting oligonucleotide functionalized with diarylcyclooctyne (DBCO) with the PD-L1-binding peptide functionalized with the azide group by a click reaction to prepare a peptide-oligonucleotide conjugate.

28. The method according to claim 27, wherein the PD-L1-binding peptide functionalized with the azide group is a compound represented by the following Formula 2, and the oligonucleotide functionalized with diarylcyclooctyne (DBCO) is a compound represented by the following Formula 3: 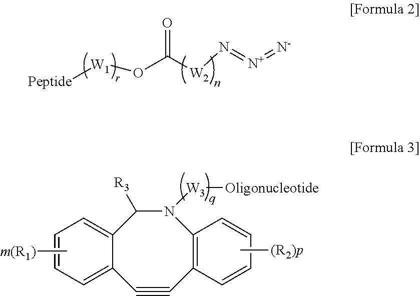
wherein R₁ to R₃ each independently represent hydrogen, a C₁-C₁₃ alkyl group, a C₁-C₆ alkoxy group, a C₆-C₁₀ aryl group, a C₃-C₁₀ cyclyl group, a C₃-C₁₀ heteroaryl group, a C₃-C₁₀ heterocyclyl group, —C(O)—(C₁-C₁₃ alkyl), —C(O)—(C₆-C₁₀ aryl), or —C(O)—(C₃-C₁₀ heteroaryl);
m, n, p, q or r each independently represents 0, 1, 2, 3 or 4;
W₁ to W₃ each independently represent —NR₃—, —NR₃CH₂—, —NR₃—C(O)—, —NR₃—C(O)—NR₄—, —NR₃—C(S)—NR₄—, —C(O)—, —C(O)CH₂—, —C(O)O—, —C(O)NR₃—, —(CH₂)—, —(CR₃R₄)—, —(CH₂)(CR₃R₄)—, —S(O)₂—, —NR₃S(O)₂—, or —S(O)₂NR₃—;
wherein the C₁-C₁₃ alkyl group, the C₃-C₁₀ cyclyl group, and the C₁-C₆ alkoxy group comprise at least one substituent selected from the group consisting of hydrogen, a hydroxyl group, a halogen group, a C₁-C₁₃ alkyl group, a C₁-C₆ alkoxy group, an amino group (—NR₃R₄), a nitro group (—N(O)₂), an amide group (—(C═O)NR₃R₄), a carboxylic acid group (—C(O)OH), a nitrile group (—CN), a urea group (—NR₃(C═O)NR₄—), a sulfonamide group (—NHS(O)₂—), a sulfide group (—S—), a sulfone group (—S(O)₂—), a phosphoryl group (—P(O)R₃R₄), a C₆-C₁₀ aryl group, a C₃-C₁₀ heteroaryl group, and a C₃-C₁₀ heterocyclyl group,
the C₆-C₁₀ aryl group, the C₃-C₁₀ heteroaryl group or the C₃-C₁₀ heterocyclyl group comprises at least one substituent selected from the group consisting of hydrogen, a hydroxyl group, a halogen group, a carbonyl group (—(C═O)R₃R₄), a C₁-C₃ alkyl group unsubstituted or substituted with a halogen or C₃-C₁₀ heterocyclyl group, a C₁-C₃ alkoxy group unsubstituted or substituted with a halogen or C₃-C₁₀ heterocyclyl group, C₆-C₁₀ phenoxy, an amino group (—NR₃R₄), a nitro group (—N(O)₂), an amide group (—(C═O)NR₃R₄), a carboxylic acid group (—C(O)OH), a nitrile group (—CN), a urea group (—NR₃(C═O)NR₄—), a sulfonamide group (—NHS(O)₂—), a sulfide group (—S—), a sulfone group (—S(O)₂—), a phosphoryl group (—P(O)R₃R₄), a C₆-C₁₀ aryl group, a C₃-C₁₀ heteroaryl group, and a C₃-C₁₀ heterocyclyl group; and
R₃ and R₄ each independently represent hydrogen, a C₁-C₆ alkyl group, a C₁-C₆ alkenyl group, a C₁-C₆ alkynyl group, a C₆-C₁₀ aryl group, a C₃-C₁₀ heteroaryl group, or a C₃-C₁₀ heterocyclyl group, or R₃ optionally comprises at least one of N, O, S, NH, C═N, C═O, —NHC(O)—, —NHC(O)NH—, —NHS(O)₂—, or SO₂ along with a nitrogen or carbon atom bound to R₄, and forms a 3- to 7-membered saturated ring that is optionally substituted with at least one of hydrogen, a C₁-C₁₃ alkyl group, a C₆-C₁₀ aryl group, a C₃-C₁₀ heteroaryl group, a hydroxyl group, a halide group, and a cyano group, and the C₃-C₁₀ heteroaryl group and the C₃-C₁₀ heterocyclyl group comprise at least one heteroatom selected from the group consisting of N, O, and S.

29. The method according to claim 28, wherein
R₁ or R₂ each independently represents hydrogen, or a C₁-C₁₃ alkyl group;
m or p each independently represents 0, 1, 2, 3 or 4;
R₃ is hydrogen, or a C₁-C₃ alkyl group;
W₁ is —(CH₂)—, —(CR₃R₄)—, or —(CH₂)(CR₃R₄)—;
r is 0, 1 or 2;
W₂ is —(CH₂)—, or —(CR₃R₄)—;
n is 1 or 2;
W₃ is —(CH₂)—, or —(CR₃R₄)—; and
q is 0, 1 or 2.

30. The method according to claim 28, wherein the click reaction in step (c) is performed in a pH 6.5-8.0 buffer solution at 700 rpm to 1,500 rpm and a temperature of 30° C. to 45° C.

31. The method according to claim 28, wherein in step (c), the oligonucleotide functionalized with diarylcyclooctyne (DBCO) and the PD-L1-binding peptide functionalized with the azide group are synthesized at a molar ratio of 1:1.5 to 1:2.5.

32. The method according to claim 28, wherein in step (C), the reaction is performed for 1 to 3 hours.

33. The method according to claim 28, wherein the PD-L1-binding peptide has a sequence of SEQ ID NO: 1.

34. The method according to claim 28, wherein the oligonucleotide is anti-miRNA-21 having a sequence of SEQ ID NO: 2 or SEQ ID NO: 3.
